(19) United States
(12) Patent Application Publication
(10) Pub. No.: US 2021/0402031 A1
(43) Pub. Date: Dec. 30, 2021

(54) PORTABLE SANITIZING CONTAINER

(71) Applicants: Theodore DeCarlo, Fort Lauderdale, FL (US); Christopher Sweeney, Fort Lauderdale, FL (US)

(72) Inventors: Theodore DeCarlo, Fort Lauderdale, FL (US); Christopher Sweeney, Fort Lauderdale, FL (US)

(21) Appl. No.: 17/169,418

(22) Filed: Feb. 6, 2021

Related U.S. Application Data
(60) Provisional application No. 63/102,550, filed on Jun. 30, 2020.

(51) Int. Cl. A61L 2/26; B65D 43/16; B65D 25/28; B65D 51/18; A61L 2/18

(52) U.S. Cl. CPC A61L 2/26; B65D 43/16; B65D 25/2897; B65D 51/18; A61L 2101/34; A61L 2/186; B65D 2251/0021; B65D 2251/0093; A61L 2/18

(57) ABSTRACT

A portable sanitizing container; the portable sanitizing container includes a container having a body, a lid having an aperture, a rubber flap covering the aperture and having a series of slits, and an inner volume housing an elongated sponge impregnated with a liquid disinfectant. The elongated sponge is configured to disperse the liquid disinfectant along an outer surface of an item to substantially disinfect an outer surface of the item. The container is configured to receive an item such as a handheld utensil through the aperture, the rubber flap, and the elongated sponge respectively and sanitize the item as the at least one item is inserted and removed from the container. The portable sanitizing container may be beneficial for use by a user to eliminate pathogens, germs and harmful bacteria on shared utensils.

### CROSS-REFERENCE TO RELATED APPLICATION(S)

The present application is related to and claims priority to U.S. Provisional Patent Application No. 63/102,550 filed Jun. 30, 2020, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of sanitizing devices of existing art and more specifically relates to sanitizing containers.

### RELATED ART

Preventing the spread of disease throughout a population is a significant problem. Diseases are often spread from one person to another person by innate objects. For example, a person infected with a disease may cough into their hands, and then touch an innate object such as a doorknob, thereby contaminating the doorknob with disease-causing germs. The hands of subsequent persons who touches that doorknob will be contaminated with the disease-causing germs left on the doorknob by the previous person and may subsequently become sick as a result thereof.

Handheld instruments or tools, such as writing instruments, are another common vehicle for spreading disease-causing germs. Recognizing that the surfaces of handheld instruments may be vehicles for passing diseases from one user to another, many individuals now carry liquid disinfectants. Individuals that use such liquid disinfectants typically dispense the disinfectant into the palm of one hand, and then spread the disinfectant over the entire surface of both hands by rubbing his or her hands together. Frequent use of such liquid disinfectants, however, may be undesirable because such disinfects can dry out and/or irritate the skin if used too frequently. Additionally, a user of such a liquid disinfectant typically uses a sufficient amount of disinfectant to disinfect his or her entire hands, even though only a small portion of a single hand may have become contaminated by touching a contaminated object. A suitable alternative solution is desired.

U.S. Pat. No. 9,155,809 to Roberts relates to a pen sterilization device. The described pen sterilization device includes pen sterilization device having a main body having a first end, a second end and a plurality of opposing sides defining a watertight reservoir for containing a sterilization agent. A pair of watertight valves are positioned along each end of the main body and function to allow ingress and egress of a pen to be cleaned. A central partition may be provided within the watertight reservoir having one or more brushes that are suspended in the sterilization agent for scrubbing a pen that is passing through the device.

### SUMMARY OF THE INVENTION

In view of the foregoing disadvantages inherent in the known sanitizing container art, the present disclosure provides a novel portable sanitizing container. The general purpose of the present disclosure, which will be described subsequently in greater detail, is to provide a portable sanitizing container. Items of common use by multiple persons, such as but not limited to writing utensils in commercial and educational environments, can be inserted into the container through an aperture, and in which the items contact the antibacterial agent.

A portable sanitizing container is disclosed herein. The portable sanitizing container destroys pathogens that may be present on items such as but not limited to writing utensils, office supplies, hand-operated tools, hair grooming accessories, and other items that can be used by multiple persons and/or in public environments. The portable sanitizing container includes a container having a body, a lid having an aperture, a rubber flap covering the aperture and having a series of slits, and an inner volume housing an elongated sponge impregnated with a liquid disinfectant. The elongated sponge includes a series of intersecting-slits through a lengthwise center. The series of intersecting-slits are in alignment with the series of slits of the rubber flap. The elongated sponge is configured to disperse the liquid disinfectant along an outer surface of an item to substantially disinfect an outer surface of the item. An exterior, bottom surface of the container of the portable sanitizing container may include means to help stabilize its placement including but are not limited to textured surface, hook-and-loop material, suction cups and/or adhesive tabs. The container is configured to receive at least one item through the aperture, the rubber flap, and the elongated sponge respectively and sanitize the item as the at least one item is inserted and removed from the container in a disinfected state.

According to another embodiment, a portable sanitizing container is also disclosed herein. The portable sanitizing container includes an elongated cylindrical container. The elongated cylindrical container comprises a body, an inner volume housing an elongated sponge impregnated with a liquid disinfectant having a slit through a lengthwise center of the elongated sponge, a first-lid positioned at a first-end of the elongated cylindrical container having an aperture, a rubber gasket member covering the aperture and having a receiving-aperture. The slit is in alignment with the receiving-aperture of the rubber gasket member. The container further includes a second-sponge impregnated with a liquid disinfectant. The second-sponge is positioned at a second-end of the elongated cylindrical container. A second-lid is provided to cover the second-sponge during non-use. The elongated cylindrical container provides a small portable version of the portable sanitizing container which is configured to receive at least one writing utensil through the aperture, the rubber gasket member, and the elongated sponge respectively and sanitize the writing utensil as the writing utensil is inserted and removed from the container. Additionally, the second-sponge may be used for sanitizing other items which cannot pass through the container such as credit cards, ATM buttons, doorknobs and phones. The shape of the present invention is not limited to cylindrical but has been described as such for exemplary means.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a portable sanitizing container, constructed and operative according to the teachings of the present disclosure.

FIG. 1 is a perspective view of a portable sanitizing container during an ‘in-use’ condition, according to an embodiment of the disclosure.

FIG. 2 is a top perspective view of the portable sanitizing container of FIG. 1 in an open-condition, according to an embodiment of the present disclosure.

FIG. 3 is a top perspective view of the portable sanitizing container of FIG. 1, according to an embodiment of the present disclosure.

FIG. 4 is a perspective view of the portable sanitizing container, according to an embodiment of the disclosure.

FIG. 5 is a perspective view of a second embodiment of the portable sanitizing container of FIG. 1, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to sanitizing containers and more particularly to a portable sanitizing container as used to improve the public health by sanitizing commonly-used and commonly-shared utensils such as pens, markers, rulers, scissors, and the like.

Generally, the portable sanitizing container provides convenient means to destroy the presence of illness-causing pathogens on commonly-used items shared by multiple users. In a primary embodiment, the portable sanitizing container includes an oval container and within that container is a liquid disinfectant and a sponge. The sponge features intersecting slits through its lengthwise center. The oval container is preferably ergonomic-shaped for easy gripping and strategic areas of the container are indented and feature a textured surface to enhance user grip. The top of the container features a removable cap with an oval, slightly subset aperture. A pliable, resilient rubber flap covers this aperture. The rubber flap features intersecting slits that are in alignment with the slits of the sponge. The rubber flap functions as a squeegee to remove access disinfectant from the utensil when said utensil is pulled out of the container. The portable sanitizing container may be made in various sizes and shapes, including specific dimensions for use with specific items. The primary embodiment of portable sanitizing container can be made in individual-use sizes.

A second embodiment is also disclosed including a smaller, portable sanitizing container that features two working ends. One end will allow for the insertion of writing instruments such as styluses and ink pens. This end features a small rubber gasket that operates like a squeegee. The opposing end comprises a second-sponge to sanitize items that cannot be inserted into the container such as credit cards, ATM buttons, doorknobs and phones. Each end will feature a removable cap. The portable sanitizing container features a slightly pliable body. As a user squeezes the body, disinfectant will saturate the second-sponge.

The portable sanitizing container can be made with various liquid disinfectant solutions, such as but not limited to ethyl alcohol and isopropyl alcohol, and in various concentrations that are effective for intended purpose. The portable sanitizing container may include various other disinfectant substances in its solution, such as but not limited to hydrogen peroxide. The portable sanitizing container may include various other materials in its solution, such as but not limited to glycerol and glycerin, and for various other purposes, such as to prevent evaporation of its alcohol base. Additionally, the portable sanitizing container may be produced in scented variations.

In other variations, the portable sanitizing container may include a caddy, bracket and/or holder used to securely mount the container upon a desk, wall, countertop and/or other surface area. The lid of the portable sanitizing container may include one (1) or more apertures and said apertures may be used to accommodate the specific shape and/or size of various utensils and other implements. The portable sanitizing container may be produced in variation as an integral feature of and/or an attachment to credit card terminals and like devices, and in which the signing stylus is stored in the container to maintain its sanitary status between users.

A method of use is as follows: a user can simply flip open the lid of portable sanitizing container and insert an item within its slotted aperture. That item will contact the sanitizing disinfectant solution upon the interior sponge and said solution will destroy any pathogens upon contact. The inserted item may then be removed. Upon removal, any residual solution clinging to the inserted item will contact the rubber flaps of the slotted aperture, returning that residual solution to the interior sponge. The user may then use the inserted item without risk of or concern about, acquiring or distributing any illness-causing pathogens. Additional sanitizing disinfectant solution may be applied to the interior sponge as needed.

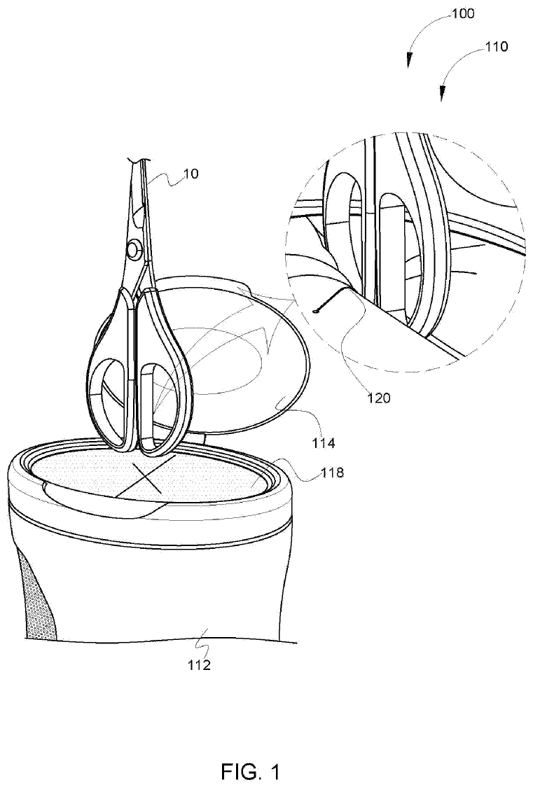

Referring now more specifically to the drawings by numerals of reference, there is shown in FIGS. 1-5, various views of a portable sanitizing container 100. The portable sanitizing container 100 destroys germs of transmittable, illness-causing viruses, including but not limited to influenza, hepatitis A, and corona virus diseases, such as but not limited to COVID-19.

FIG. 1 shows a portable sanitizing container 100 during an ‘in-use’ condition, according to an embodiment of the present disclosure. Here, the portable sanitizing container 100 may be beneficial for use by a user to eliminate pathogens, germs and harmful bacteria on shared utensils. As illustrated, in a first embodiment, the portable sanitizing container 100 may include a container 110 having a body 112, a lid 114 having an aperture 116, a rubber flap 118 covering the aperture 116 and having a series of slits 120, and an inner volume 122 housing an elongated sponge 124 impregnated with a liquid disinfectant. The elongated sponge 124 includes a series of intersecting-slits 126 through a lengthwise center. The series of intersecting-slits 126 are in alignment with the series of slits 120 of the rubber flap 118.

As shown, the rubber flap 118 is pliable and configured to contour to an exterior surface of an item 10 as the item 10 passes therethrough. The elongated sponge 124 is configured to disperse the liquid disinfectant along the exterior surface of the item 10 to substantially disinfect an exterior surface of the item 10. The container 110 is configured to receive an item 10 such as a handheld utensil through the aperture 116, the rubber flap 118, and the elongated sponge 124 respectively and sanitize the item 10 as the item 10 is inserted and removed from the container 110.

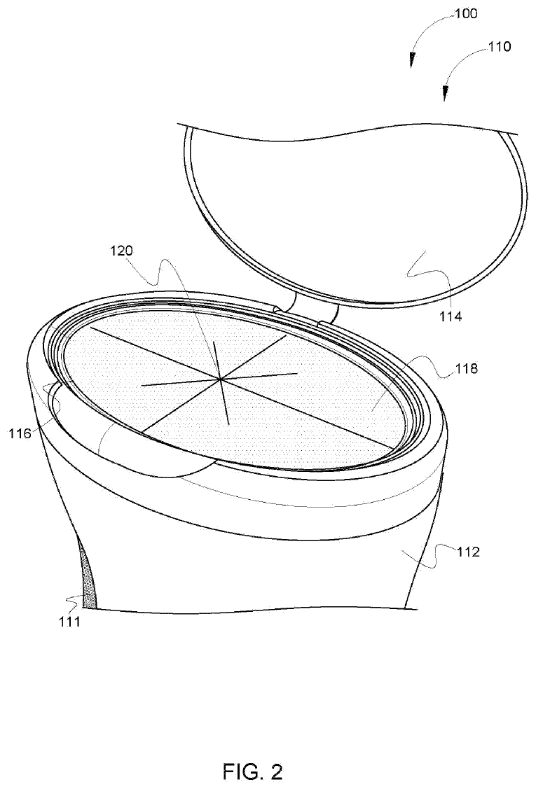

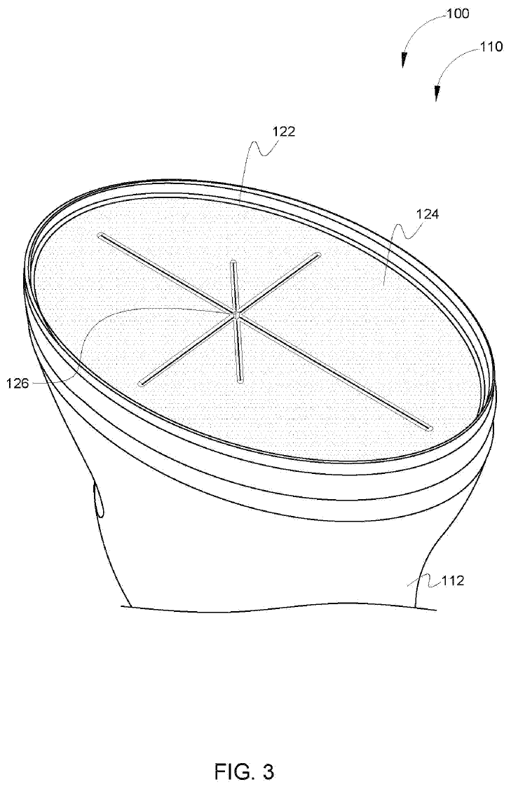

FIGS. 2-3 show the portable sanitizing container 100 of FIG. 1, according to an embodiment of the present disclosure. As above, the portable sanitizing container 100 may include the container 110 including the body 112, the lid 114 having an aperture 116, and the rubber flap 118 covering the aperture 116. The lid 114 includes a flip top cap which is hingedly coupled to the lid 114 and configured to open and close. The lid 114 may be removable from the body 112 of the container 110. As shown, the rubber flap 118 includes the series of slits 120. In a preferred version of the first embodiment, the container 110 preferably comprises a slightly oval, substantially cylindrical profile however other profiles/shapes may be employed. Additionally, the container 110 comprises an indented side portion having a textured gripping surface 111. The container 110 comprises a container-depth sized and arranged to fully receive at least one item 10 for disinfection of at least one item 10. The portable sanitizing container 100 destroys bacteria, viruses, and other pathogens that cause illnesses upon contact.

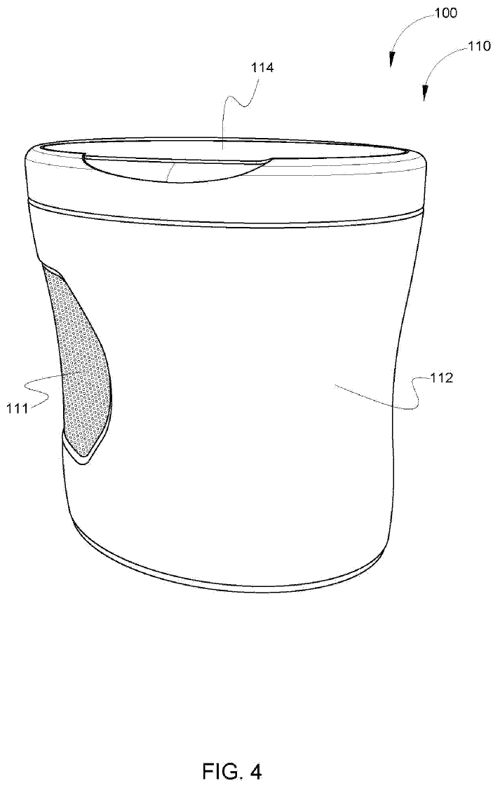

FIG. 4 is a perspective view of the portable sanitizing container 100 of FIG. 1, according to an embodiment of the present disclosure. The container 110 of the portable sanitizing container 100 may comprise an approximate six and one-half inch (6½″) height, six-inch (6″) length and a four-inch (4″) width. Items 10 inserted within the portable sanitizing container 100 make contact with the liquid disinfectant and the elongated sponge 124 to ensure they are thoroughly cleaned. The liquid disinfectant kills pathogens and the elongated sponge 124 ensures the entire item 10 is covered and scrubbed. The elongated sponge 124 may include a synthetic cellulose sponge material or other suitable absorbent material. The liquid disinfectant may comprise an ethyl alcohol for seventy percent (70%) of its solution. The remaining portion of the liquid disinfectant may include glycerol and distilled water. The liquid disinfectant may be reapplied to the elongated sponge of portable sanitizing container 100 as needed.

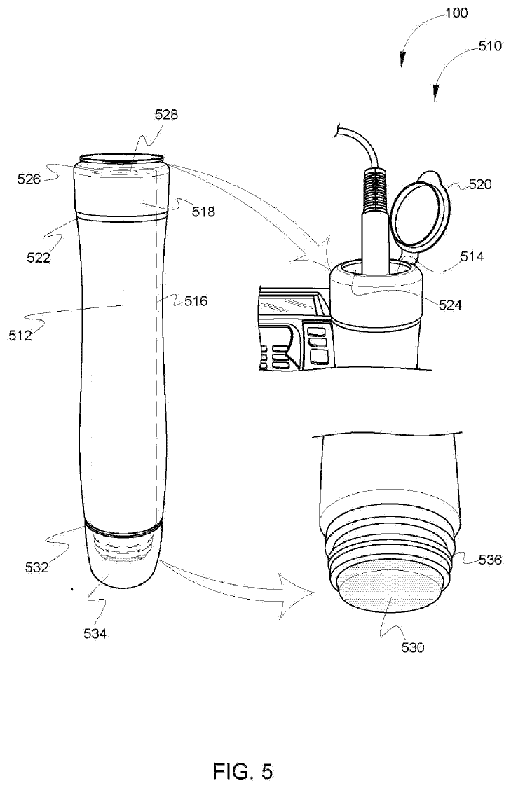

FIG. 5 is a perspective view of a second embodiment of the portable sanitizing container 100 of FIG. 1, according to an embodiment of the present disclosure. The portable sanitizing container 100 includes an elongated cylindrical container 510. The elongated cylindrical container 510 comprises a body 512, an inner volume 514 housing an elongated sponge 516 impregnated with a liquid disinfectant having a slit 518 through a lengthwise center of the elongated sponge 516, a first-lid 520 positioned at a first-end 522 of the elongated cylindrical container 510 having an aperture 524, a rubber gasket member 526 covering the aperture 524 and having a receiving-aperture 528. The slit 518 is in alignment with the receiving-aperture 528 of the rubber gasket member 526.

The elongated cylindrical container 510 further includes a second-sponge 530 impregnated with a liquid disinfectant. The second-sponge 530 is positioned at a second-end 532 of the elongated cylindrical container 510. A second-lid 534 is provided to cover the second-sponge 530 during non-use. The elongated cylindrical container 510 provides a small portable version of the portable sanitizing container 100 which is configured to receive at least one writing utensil through the aperture 524, the rubber gasket member 526, and the elongated sponge 516 respectively and sanitize the writing utensil as the writing utensil is inserted and removed from the container. Additionally, the second-sponge 530 may be used for sanitizing other items which cannot pass through the container such as credit cards, ATM buttons, doorknobs and phones.

As shown in FIG. 5, the container includes the elongated sponge 516 and the second-sponge 530 positioned at the second-end 532 of the elongated cylindrical container 510. The second-sponge 530 may comprise a flat circular profile or in alternative embodiments, may include a marker nib-like profile. The first-lid 520 is hingedly connected to the elongated cylindrical container 510 and configured to open and alternatively close. As illustrated, the second-lid 534 of the container may be removable. The second-end 532 comprises a threaded section 536 in a preferred embodiment which is configured to mate with the second-lid 534.

The receiving-aperture 528 of the rubber gasket member 526 is sized and arranged to receive a writing utensil such as a pen, stylus, pencil or the like therethrough. The rubber gasket member 526 is pliable and contours to an exterior surface of a writing utensil as the writing utensil passes therethrough. The elongated cylindrical container 510 comprises a depth sized and arranged to fully receive the writing utensil for disinfection of the writing utensil. The body 512 of the elongated cylindrical container 510 is semi-pliable. Compression of the body 512 supplies the liquid disinfectant to the second-sponge 530.

In a most preferred version of the second embodiment of the portable sanitizing container 100, the elongated cylindrical container 510 comprises an approximate six-inch (6″) length by one-inch (1″) diameter. Again, as above, other shapes and profiles may be used. When in need of using a public-access pen, a user may insert the pen within the elongated cylindrical container 510. When in need to use a touch pad, such as at an ATM or at a credit card panel, the user can remove the second-lid 534 and use its exposed second-sponge 530 to disinfect the credit card panel(s).

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application

What is claimed is new and desired to be protected by Letters Patent is set forth in the appended claims:

1. A portable sanitizing container, the portable sanitizing container comprising:
a container having;
a body;
a lid having;
an aperture;
a rubber flap covering said aperture and having a series of slits;
an inner volume housing;
an elongated sponge impregnated with a liquid disinfectant and having a series of intersecting-slits through a lengthwise center of said elongated sponge, said series of intersecting-slits being in alignment with said series of slits of said rubber flap;
wherein said container is configured to receive at least one item through said aperture, said rubber flap, and said elongated sponge respectively and sanitize said at least one item as said at least one item is inserted and removed from said container; and
wherein said elongated sponge is configured to disperse said liquid disinfectant along an outer surface of said at least one item to substantially disinfect said at least one item.

2. The portable sanitizing container of claim 1, wherein said lid includes a flip top cap which is hingedly coupled to said lid.

3. The portable sanitizing container of claim 1, wherein said container comprises a slightly oval, substantially cylindrical profile.

4. The portable sanitizing container of claim 3, wherein said container comprises an indented side portion.

5. The portable sanitizing container of claim 4, wherein said indented side portion includes a textured gripping surface.

6. The portable sanitizing container of claim 1, wherein said rubber flap is pliable and configured to contour to an exterior surface of said at least one item as said at least one item passes therethrough.

7. The portable sanitizing container of claim 1, wherein said container comprises a container-depth sized and arranged to fully receive said at least one item for disinfection of said at least one item.

8. The portable sanitizing container of claim 1, wherein said at least one item includes handheld utensils.

9. The portable sanitizing container of claim 1, wherein said lid is removable from said body of said container.

10. A portable sanitizing container, the portable sanitizing container comprising:
an elongated cylindrical container having;
a body;
a first-lid positioned at a first-end of said elongated cylindrical container having;
an aperture;
a rubber gasket member covering said aperture and having a receiving-aperture;
an inner volume housing
an elongated sponge impregnated with a liquid disinfectant and having a slit through a lengthwise center of said elongated sponge, said slit being in alignment with said receiving-aperture of said rubber gasket member;
a second-sponge impregnated with a liquid disinfectant and being positioned at a second-end of said elongated cylindrical container; and
a second-lid configured to cover said second-sponge during non-use; and
wherein said elongated cylindrical container is configured to receive at least one writing utensil through said aperture, said rubber gasket member, and said elongated sponge respectively and sanitize said at least one writing utensil as said at least one writing utensil is inserted and removed from said container.

11. The portable sanitizing container of claim 10, wherein said second-sponge comprises a flat circular profile.

12. The portable sanitizing container of claim 10, wherein said second-sponge comprises a marker nib-like profile.

13. The portable sanitizing container of claim 10, wherein said first-lid is hingedly connected to said elongated cylindrical container.

14. The portable sanitizing container of claim 13, wherein said receiving-aperture is sized and arranged to receive said at least one writing utensil therethrough.

15. The portable sanitizing container of claim 14, wherein said body of said elongated cylindrical container is semi-pliable.

16. The portable sanitizing container of claim 15, wherein compression of said body supplies said liquid disinfectant to said second-sponge.

17. The portable sanitizing container of claim 14, wherein said elongated cylindrical container comprises a depth sized and arranged to fully receive said at least one writing utensil for disinfection of said at least one writing utensil.

18. The portable sanitizing container of claim 1, wherein said second-lid is removable.

19. The portable sanitizing container of claim 18, wherein said second-end comprises a threaded section configured to mate with said second-lid.

20. A portable sanitizing container, the portable sanitizing container comprising:
a container having;
a body;
a lid having;
an aperture;
a rubber flap covering said aperture and having a series of slits;
an inner volume housing;
an elongated sponge impregnated with a liquid disinfectant and having a series of intersecting-slits through a lengthwise center of said elongated sponge, said series of intersecting-slits being in alignment with said series of slits of said rubber flap;
wherein said container comprises a slightly oval, substantially cylindrical profile;
wherein said container comprises a container-depth sized and arranged to fully receive at least one item for disinfection of said at least one item;
wherein said container comprises an indented side portion;
wherein said indented side portion includes a textured gripping surface;
wherein said lid includes a flip top cap which is hingedly coupled to said lid;
wherein said lid is removable from said body of said container;
wherein said rubber flap is pliable and configured to contour to an exterior surface of said at least one item as said at least one item passes therethrough;
wherein said container is configured to receive at least one item through said aperture, said rubber flap, and said elongated sponge respectively and sanitize said at least one item as said at least one item is inserted and removed from said container;
wherein said elongated sponge is configured to disperse said liquid disinfectant along an outer surface of said at least one item to substantially disinfect said at least one item; and
wherein said at least one item includes handheld utensils.
